(19) United States
(12) Patent Application Publication
(10) Pub. No.: US 2004/0002763 A1
(43) Pub. Date: Jan. 1, 2004

(54) Spinal disc anulus occlusion device and method of use

(75) Inventors: Anthony C. Phillips, Eden Prairie, MN (US); John Gainor, White Bear Township, MN (US); Orson James May, Macon, GA (US); Britt K. Norton, Eden Prairie, MN (US)

Correspondence Address: Timothy A. Czaja, Dicke, Billig & Czaja, P.A., 701 Building, Suite 1250, 701 Fourth Avenue South, Minneapolis, MN, 55415, US

(73) Assignee: Raymedica, Inc.

(21) Appl. No.: 10/185,832

(22) Filed: Jun. 27, 2002

(51) Int. Cl. A61F 2/44

(52) U.S. Cl. 623/17.16

(57) ABSTRACT

An occlusion device for occluding a defect in a spinal disc anulus having interior and exterior surfaces, and defining an internal cavity. The occlusion device includes a first member, a second member, and a connector. The first member is configured for deployment within the internal cavity and placement against the interior surface of the anulus. The second member is configured for placement against the exterior surface of the anulus. Finally, the connector connects the first and second members and is preferably adapted to provide an adjustable spacing therebetween. With this construction, the device is configured such that upon final deployment, the first and second members are rigidly secured against the anulus, at opposite surfaces thereof, in a region of the defect via the connector.

### BACKGROUND OF THE INVENTION

The present invention relates to a device for occluding a defect through a spinal disc anulus. More particularly, it relates to an occlusion device adapted to provide a strong mechanical closure or barrier for a spinal disc anulus defect.

The vertebral spine is the axis of the skeleton upon which all of the body parts “hang”. In humans, the normal spine has seven cervical, twelve thoracic and five lumbar segments. The lumbar segments sit upon a sacrum, which then attaches to a pelvis, in turn supported by hip and leg bones. The bony vertebral bodies of the spine are separated by intervertebral discs, which act as joints, but allow known degrees of flexion, extension, lateral bending and axial rotation.

The typical vertebra has a thick interior bone mass called the vertebral body, and a neural (vertebral) arch that arises from a posterior surface of the vertebral body. Each neural arch combines with the posterior surface of the vertebral body and encloses a vertebral foramen. The vertebral foramina of adjacent vertebrae are aligned to form a vertebral canal, through which the spinal sac, cord and nerve rootlets pass. The portion of the neural arch that extends posteriorly and acts to protect a posterior side of the spinal cord is known as the lamina. Projecting from the posterior region of the neural arch is a spinous process. The central portions of adjacent vertebrae are separated and supported by an intervertebral disc.

The intervertebral disc primarily serves as a mechanical cushion between the vertebral bones, permitting controlled motions within vertebral segments of the axial skeleton. The normal disc is a unique, mixed structure, comprised of three component tissues: The nucleus pulposus (“nucleus”), the anulus fibrosus (“anulus”), and two opposing vertebral end plates. The two vertebral end plates are each composed of thin cartilage overlying a thin layer of hard, cortical bone which attaches to the spongy, richly vascular, cancellous bone of the vertebral body. The end plates thus serve to attach adjacent vertebrae to the disc. In other words, a transitional zone is created by the end plates between the malleable disc and the bony vertebrae.

The anulus of the disc is a tough, outer fibrous ring that binds together adjacent vertebrae. This fibrous portion, which is much like a laminated automobile tire, is generally about 10 to 15 millimeters in height and about 15 to 20 millimeters in thickness. The fibers of the anulus consist of 15 to 20 overlapping multiple plies, and are inserted into the superior and inferior vertebral bodies at roughly a 30 degree angle in both directions. This configuration particularly resists torsion, as about half of the angulated fibers will tighten when the vertebrae rotate in either direction, relative to each other. The laminated plies are less firmly attached to each other.

Immersed within the anulus, positioned much like the liquid core of a golf ball, is the nucleus. The anulus and opposing end plates maintain a relative position of the nucleus in what can be defined as a nucleus cavity. The healthy nucleus is largely a gel-like substance having high water content, and similar to air in a tire, serves to keep the anulus tight yet flexible. The nucleus-gel moves slightly within the anulus when force is exerted on the adjacent vertebrae with bending, lifting, etc.

Under certain circumstances, an anulus defect (or anulotomy) can arise that requires surgical attention. These anulus defects can be naturally occurring, surgically created, or both. A naturally occurring anulus defect is typically the result of trauma or a disease process, and may lead to a disc herniation. A disc herniation occurs when the anulus fibers are weakened or torn and the inner tissue of the nucleus becomes permanently bulged, distended, or extruded out of its normal, internal anular confines. The mass of a herniated or “slipped” nucleus can compress a spinal nerve, resulting in leg pain, loss of muscle control, or even paralysis.

Where the naturally occurring anulus defect is relatively minor and/or little or no nucleus tissue has escaped from the nucleus cavity, satisfactory healing of the anulus may be achieved by immobilizing the patient for an extended period of time. A more practical solution would be artificially obstructing or occluding the defect with an auxiliary device. Unfortunately, an effective anulus defect occluder able to maintain its position relative to an even minor anulus defect has not heretofore been developed.

A more problematic anulus defect concern arises in the realm of anulotomies encountered as part of a surgical procedure performed on the disc space. As a starting point, bed rest alone cannot adequately heal many disc herniations, such that a more traumatic surgical intervention is required. Alternatively, with discal degeneration, the nucleus loses its water binding ability and deflates, as though the air had been let out of a tire. Subsequently, the height of the nucleus decreases, causing the anulus to buckle in areas where the laminated plies are loosely bonded. As these overlapping laminated plies of the anulus begin to buckle and separate, either circumferential or radial anular tears may occur, which may contribute to persistent and disabling back pain. Adjacent, ancillary spinal facet joints will also be forced into an overriding position, which may create additional back pain.

In many cases, to alleviate pain from degenerated or herniated discs, the nucleus is removed and the two adjacent vertebrae surgically fused together. While this treatment alleviates the pain, all discal motion is lost in the fused segment. Ultimately, this procedure places greater stress on the discs adjacent the fused segment as they compensate for the lack of motion, perhaps leading to premature degeneration of those adjacent discs. A more desirable solution entails replacing, in part or as a whole, the damaged nucleus with a suitable prosthesis having the ability to complement the normal height and motion of the disc while stimulating the natural disc physiology.

The first prostheses embodied a wide variety of ideas, such as ball bearings, springs, metal spikes and other perceived aids. These prosthetic discs were designed to replace the entire intervertebral disc space, and were large and rigid. Beyond the questionable efficacy of these devices is the inherent difficulties encountered during implantation. Due to their size and inflexibility, these first generation devices require an anterior implantation approach as the barriers presented by the lamina and, more importantly, the spinal cord and nerve rootlets during posterior implantation, could not be avoided. Recently, smaller and more flexible prosthetic nucleus devices have been developed. With the reduction in prosthesis size, the ability to work around the spinal cord and nerve rootlets during posterior implantation has become possible.

Generally speaking, these reduced size prostheses are intended to serve as a replacement for the natural nucleus. In other words, the anulus and end plates remain intact, and the prosthesis is implanted within the nucleus cavity. In order to implant a prosthesis within the nucleus cavity, an appropriately sized passageway through the anulus (i.e., anulotomy) must exist. The requisite anulus defect can be surgically imparted as part of the surgical implantation procedure, or the naturally occurring anulus defect that caused or resulted from the discal failure may be large enough for passage of the prosthesis. One pre-implant anulotomy technique entails complete removal of a plug of tissue from the anulus via an incision created by a scalpel, punch or similar tool. Entire removal of an anulus segment is highly traumatic, and limits the ability of the anulus to properly heal. Attempts to reattach the anulus plug have been unavailing in that properly orienting and securing of the anulus plug with a suture has proven difficult at best. Alternatively, a flap can be imparted into the anulus tissue. This technique overcomes the reattachment problems associated with the anulus plug approach. Unfortunately, however, the thickness of the anulus requires formation of a relatively large flap, therefore increasing anulus trauma. Further, it may be difficult to retain the flap in a retracted position throughout the implantation procedure. A third, more viable procedure is to dilate a small opening or incision in the anulus to a size sufficient for prosthesis implantation. The overlapping, plied nature of the anulus tissue renders the anulus highly amenable to incision dilation.

Regardless of the anulotomy technique, the resulting anulus defect may lead to post-implant complications. The anulus tissue will, in theory, regenerate or naturally repair the defect over time. However, substantial scar tissue formation will not occur for a significant period of time, and requires that forces on the spinal tract be minimized (i.e., that the patient be immobilized). For virtually all patients, this is impossible to achieve. Instead, within several days of the implantation procedure, the patient must move about, thereby placing forces on the disc space. Because the anulus defect has not healed, it cannot readily prevent the prosthetic nucleus, or portions thereof (depending upon the particular prosthesis construction), from migrating back through the anulus defect. Even if this opening is closed via sutures following implant, various forces acting upon the disc space have the potential to overcome the resistance provided by the sutures and “push” the prosthesis back through the anulus opening. A more preferable solution would be the provision of an auxiliary device that serves to not only occlude the surgically-created anulus defect, but also rigidly resists explant of the prosthesis, or portions thereof, back through the opening. Unfortunately, and as previously described, such a device has not heretofore been developed.

Spinal disc anulus defects occur both naturally and as part of a surgical procedure. Currently accepted techniques of suturing the defect closed are of minimal value in light of the forces normally encountered by the disc space. Even more problematic is the inability to protect against explant of a prosthetic spinal disc nucleus otherwise implanted through a surgical-imparted anulus defect. Therefore, a need exists for a spinal anulus defect occlusion device capable of effectuating anulus repair and providing a strong mechanical closure/barrier required for successful prosthetic disc nucleus implantation.

### SUMMARY OF THE INVENTION

One aspect of the present invention relates to an occlusion device for occluding a defect in a spinal disc anulus. In this regard, the anulus has an interior surface and an exterior surface, and defines an internal cavity. With this in mind, the occlusion device includes a first member, a second member, and a connector. The first member is configured for deployment within the internal cavity and placement against the interior surface of the anulus. Conversely, the second member is configured for placement against the exterior surface of the anulus. Finally, the connector connects the first and second members and is configured to provide an adjustable spacing therebetween. With this construction, the device is configured such that upon final deployment, the first and second members are secured against the anulus, at opposite surfaces thereof, in a region of the defect via the connector. The adjustable spacing afforded by the connector facilitates a more rigid securement to the anulus. In one preferred embodiment, the first member defines a generally convex anulus contact face, whereas the second member defines a generally concave anulus contact face, such that the first and second member more readily conform to a shape of the anulus.

Another aspect of the present invention relates to a method of occluding a defect in a spinal disc anulus. Once again, the anulus includes an inner surface and an outer surface, and defines an internal cavity. With this in mind, the method includes deploying a first member within the internal cavity of the spinal disc anulus. A second member is deployed at an outer surface of the anulus in a region of the defect. A connector is extended through the defect and connects the first and second member. Finally, the first member is secured relative to the second member via the connector such that the first and second members engage the inner and outer surfaces, respectively, of the anulus. Further, once secured, the first and second members, the connector and the anulus combine to rigidly resist transverse expulsion of the first member back through the defect. In one preferred embodiment, the step of securing the first member relative to the second member entails drawing the first and second members toward one another via the connector, such that the anulus tissue is pinched between the first and second members.

Yet another aspect of the present invention relates to a method of implanting a prosthetic spinal disc nucleus into a nucleus cavity defined by an anulus. In this regard, the anulus defines an inner surface and outer surface. With this in mind, the method includes creating an opening through the anulus. A prosthetic spinal disc nucleus is inserted into the nucleus cavity through the opening. A first occlusion member is deployed into the nucleus cavity in a region of the opening. A second occlusion member is deployed at an outer surface of the anulus in the region of the opening. A connector is extended through the opening and connects the first and second members. Finally, the first member is secured relative to the second member via the connector such that the first and second members engage the inner and outer surfaces, respectively, of the anulus. Further, this securement between the first and second members rigidly resist expulsion of the prosthetic spinal disc nucleus back through the opening.

### BRIEF DESCRIPTION OF THE DRAWINGS

FIG. 1 is a posterior view of a spinal segment including a discal area within the which the device and method of the present invention are useful;

FIG. 2 is an enlarged top, sectional view of the disc space of FIG. 1;

FIG. 3 is a top, sectional view of the disc space of FIG. 2 in conjunction with a deployed occlusion device in accordance with the present invention;

FIG. 4 is an exploded, perspective view of the occlusion device of FIG. 3;

FIGS. 5A-5C illustrate a method of deploying the occlusion device of FIG. 4;

FIGS. 6A and 6B are a side view of alternative embodiment components of the device of FIG. 4;

FIG. 7 is a top, sectional view of a portion of the disc space of FIG. 2 in conjunction with a deployed, alternative embodiment occlusion device in accordance with the present invention;

FIG. 8A is a top, sectional view of a portion of the disc space of FIG. 2 in conjunction with a deployed, alternative embodiment occlusion device in accordance with the present invention;

FIG. 8B is a posterior view of a spinal segment and illustrates deployment of the occlusion device of FIG. 8A;

FIG. 9A is a perspective view of an alternative embodiment occlusion device in accordance with the present invention upon final deployment;

FIG. 9B illustrates the occlusion device of FIG. 9A in a delivery state; and

FIG. 10 is a perspective view of an alternative embodiment occlusion device in accordance with the present invention.

### DESCRIPTION OF THE PREFERRED EMBODIMENTS

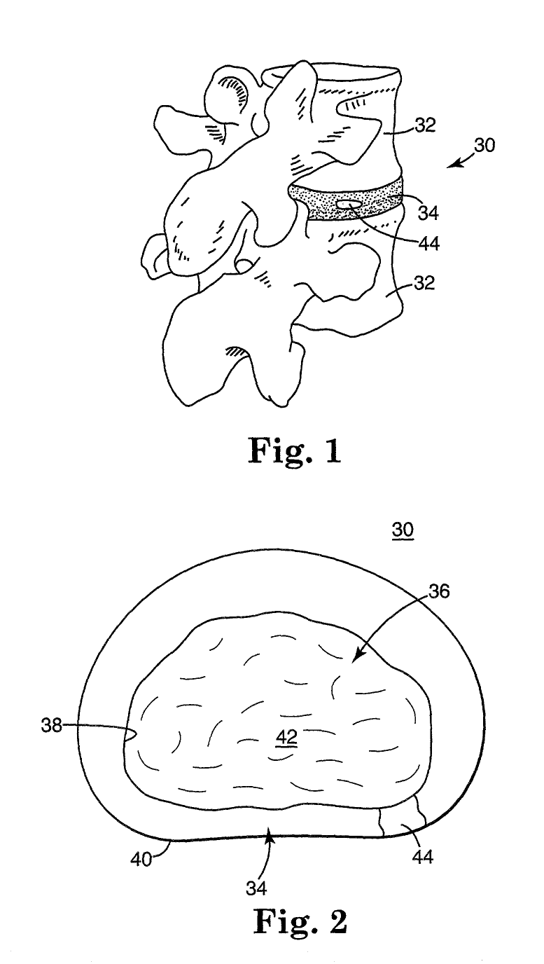

The present inventions relates to an occlusion device for occluding a defect in a spinal disc anulus. As a point of reference, FIGS. 1 and 2 depict a disc space 30 for which the device and method of the present invention are applicable. The disc space 30 separates adjacent vertebrae 32 and includes an anulus 34 and a nucleus 36 (shown in FIG. 2). The anulus 34 defines an inner surface 38 and an outer surface 40. Further, the anulus 34, in combination with end plates (not shown) associated with the opposing vertebrae 32, defines a nucleus cavity 42 (referenced generally in FIG. 2) within which the nucleus 36 is contained. With the illustrations of FIGS. 1 and 2, a defect 44 is formed in the anulus 34. The defect 44 can be naturally occurring, such as a tear or other trauma to the anulus 34. Alternatively, or in addition, the defect 44 can be surgically created as part of a disc space repair procedure. For example, and as described in greater detail below, a small incision may be formed in the anulus 34 to facilitate implantation of a prosthesis (not shown) into the nucleus cavity 42. Thus, although the defect 44 is illustrated in FIGS. 1 and 2 as being relatively uniform, the defect 44 can assume a wide variety of shapes and sizes.

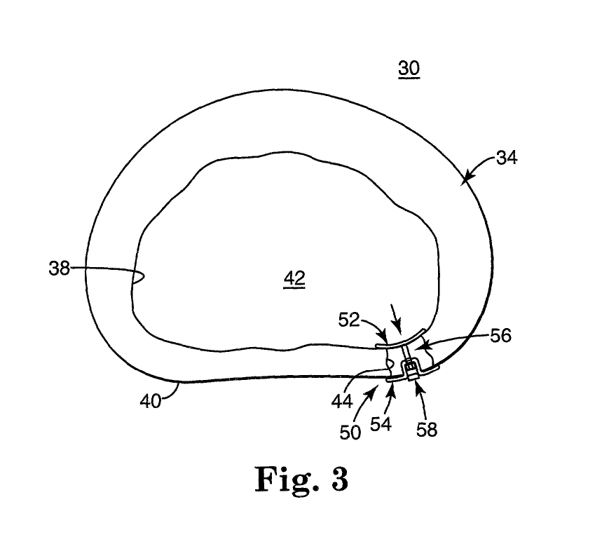

With the above definitions in mind, FIG. 3 illustrates one preferred embodiment of an occlusion device 50 in accordance with the present invention deployed to the above-described disc space 30. For ease of illustration, the nucleus 36 has been removed from the view of FIG. 3. With this in mind, the occlusion device 50 is configured to satisfy the unique constraints presented by the disc space 30, and includes a first member 52, a second member 54, a connector 56, and a cap 58. The various components are described in greater detail below. In general terms, however, the first member 52 is secured to the second member 54 by the connector 56. Upon final deployment, the first member 52 engages the inner surface 38 of the anulus 34. Similarly, the second member 54 engages the outer surface 40 of the anulus 34. The connector 56 extends through the defect 44 and rigidly secures the first and second members 52, 54 to the anulus 34. Finally, the cap 58 is secured to the second member 54, and covers the connector 56 relative to an exterior of the anulus 34. Rigid engagement of the occlusion device 50 relative to the anulus 34 serves to prevent transverse displacement or movement of the first member 52 outwardly through the defect 44.

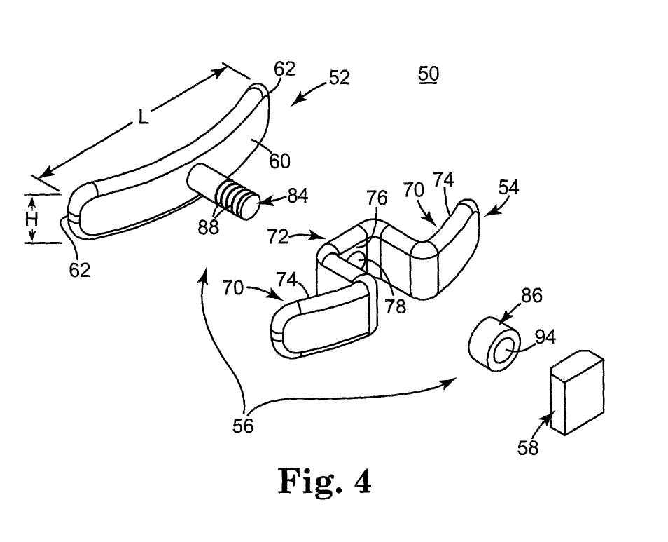

With additional reference to FIG. 4, the first member 52 is preferably in the form of a rigid, elongated plate that defines an anulus contact face 60 extending between opposing ends 62. The anulus contact face 60 is generally convex, conforming with a general shape of the inner surface 38 of the anulus 34. A length (“L” in FIG. 4) of the first member 52 can vary depending upon the particular application (e.g., generally longer for use in maintaining a previously-implanted prosthetic disc nucleus as compared to general anulotomy repair), but is preferably on the order of 10-30 mm in length. It has surprisingly been found that providing the first member 52 with a length in the range of 10-30 mm facilitates relatively easy insertion into the nucleus cavity 42 while providing sufficient surface area for encompassing most normally encountered anulus defects and anulus engagement as described below. Further, the first member 52 has a height (“H” in FIG. 4) that approximates an expected height of the anulus 34. In a preferred embodiment, the first member 52 has a height on the order of 5-15 mm. Although not illustrated in FIG. 4, in one alternative embodiment, the opposing ends 62 are preferably relatively sharp to assist in engaging or “biting” into the anulus 34 upon final deployment. Finally, the first member 52 is preferably formed from a strong, biocompatible material, including metals or plastics, such as polyetheretherketone (PEEK), carbon fiber composite, etc. These preferred dimensions and materials have surprisingly been found to facilitate transversely rigid engagement between the first member 52 and the anulus 34 as described below.

The second member 54 is preferably formed as an elongated, rigid body. In this regard, the second member 54 includes opposing outer regions 70 and a central region 72. The opposing outer regions 70 combine to define an anulus contact face 74 that, in a preferred embodiment, is generally concave, thereby conforming generally with the shape of the outer surface 40 of the anulus 34. The central region 72 projects distally relative to the opposing outer regions 70, thereby defining an indentation. In other words, the second member 54 includes an outer face 76 that forms the indentation at the central region 72 relative to the opposing outer regions 70. As described in greater detail below, the central region 72 is preferably sized to project within the defect 44 upon deployment of the second member 54 to the outer surface 40 of the anulus 34, providing a self-centering feature and preventing movement of the device 50 following deployment. Finally, the central region 72 includes a passage 78 sized to receive a portion of the connector 56 as described below.

Similar to the first member 52, the second member 54 is preferably formed of a relatively rigid, biocompatible material such as PEEK, carbon fiber composite, etc. Further, the second member 54 preferably has height and width (or length) dimensions similar to those of the first member 52. Alternatively, however, and because the second member 54 is not deployed within the nucleus cavity 42, the second member 54 can be slightly larger than the first member 52.

The connector 56 preferably includes a post 84 and a coupler 86. The post 84 extends proximally from the anulus contact face 60 of the first member 52. In this regard, the post 84 is preferably centered relative to a length of the first member 52. Further, the post 84 is sized to be slidably received within the passage 78 of the second member 54, and preferably forms a series of notches 88. As described below, the notches 88 facilitate locking engagement between the post 84 and the coupler 86. As such, other coupling designs, such as threads, can be employed. Regardless, the post 84 is preferably formed of a relatively rigid, biocompatible material such as PEEK, carbon fiber composite, etc., and defines a length (or extension from the anulus contact face 60) commensurate with an expected transverse width of the anulus 34. Thus, in one preferred embodiment, the post 84 has a length in the range of approximately 5-15 mm.

The coupler 86 is a ring-like component, and defines a central hole 94 sized to slidably receive the post 84. Further, the coupler 86 preferably defines one or more internal, deflectable fingers (not shown) that extend radially within the central hole 94. The fingers are configured to selectively engage each of the notches 88 as the coupler 86 is forced along the post 84. More particularly, the fingers facilitate locking of the coupler 86 relative to the post 84 as the fingers engage a respective one of the notches 88. That is to say, in a preferred embodiment, the fingers allow the coupler 86 to be slid distally along the post 84, but prevent proximal (or rearward) movement of the coupler 86 relative to the post 84 once the fingers have engaged a particular one of the notches 88, such that the coupler 86 serves as a locking component. Alternatively, a wide variety of other locking techniques, such as threads, can be employed.

Finally, the cap 58 is configured to selectively engage the second member 54 at the central region 72. For example, the outer face 76 of the central region 72 can form grooves sized to frictionally receive opposing edges of the cap 58 in a snap-fit relationship. Alternatively, the cap 58 and/or the second member 54 can incorporate other configurations that facilitate assembly of the cap 58 to the second member 54. Regardless, the cap 58 serves to cover the indentation otherwise defined by the central region 72 upon final deployment, and is preferably configured to mesh with a profile of the second member 54.

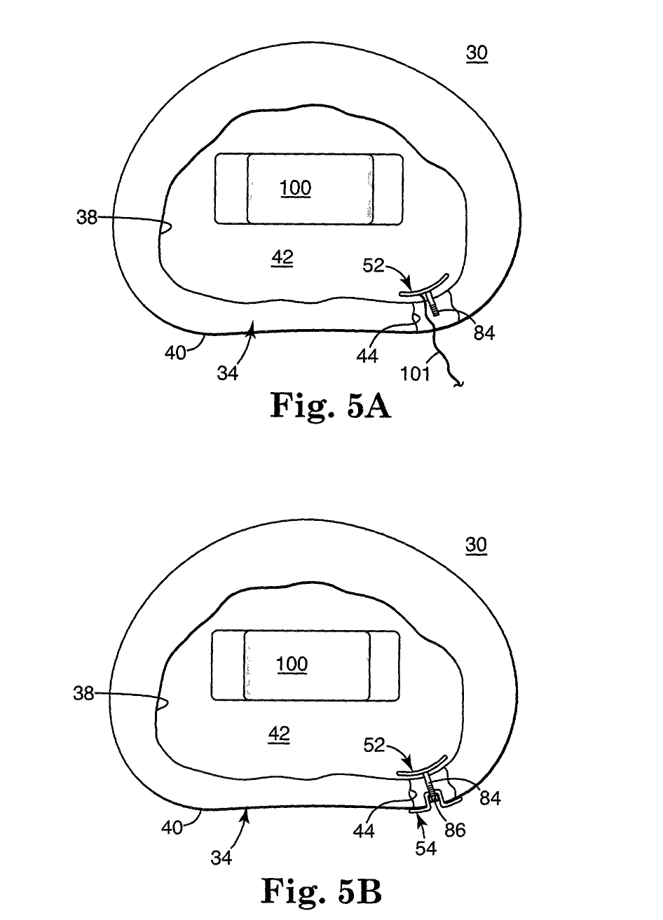

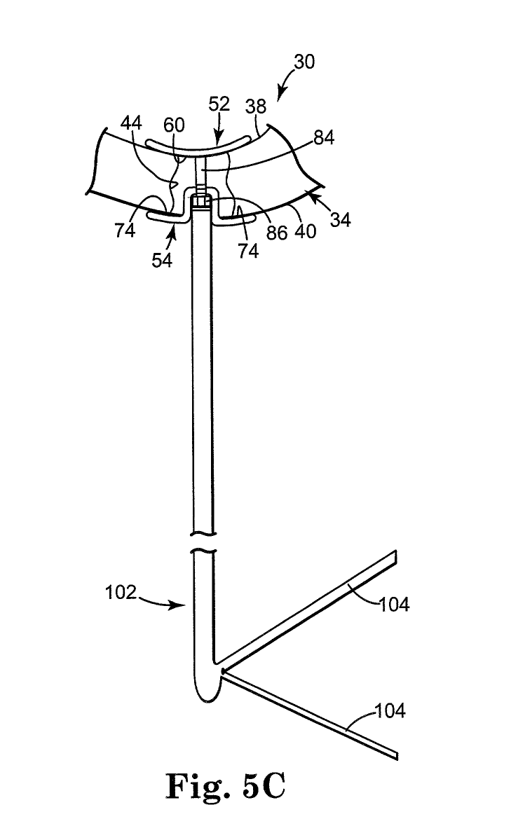

Deployment of the occlusion device 50 to a disc space 30 is best described with reference to FIGS. 5A-5C. As a point of reference, the deployment methodology associated with FIGS. 5A-5C is provided in conjunction with a prosthetic spinal disc nucleus implantation procedure. In particular, the defect or passage 44 is imparted through the anulus 34. In one preferred embodiment, some or all of the nucleus material 36 (FIG. 1) is removed from the nucleus cavity 42. A prosthetic spinal disc nucleus 100 (shown generally in FIGS. 5A and 5B) is then implanted into the nucleus cavity 42 as shown in FIG. 5A. It will be understood that the prosthetic nucleus 100 illustrated in FIGS. 5A and 5B is but one example of an acceptable device and is in no way limiting; a prosthetic nucleus can have a wide variety of shapes, sizes, materials, constructions, etc., as is known in the art.

Following implantation of the prosthetic spinal disc nucleus 100, the first member 52 is inserted through the defect 44 and positioned within the nucleus cavity 42 as shown. To facilitate handling and temporary retention of the first member 52 during this insertion or deployment operation, a suture 101 or other removable component can be connected to the first member 52 and extended outwardly from the nucleus cavity 42 via the defect 44. Alternatively, the post 84 provides a convenient surface for handling the first member 52 during the deployment procedure, such that the suture 101 or other removable component is not required.

With the first member 52 properly positioned, the second member 54 is then deployed relative to the outer surface 40 of the anulus 34 as shown in FIG. 5B. In particular, the second member 54 is positioned such that the post 84 extends through the passage 78 (FIG. 4) of the second member 54. The coupler 86 is then placed over the post 84 as shown. Notably, in FIG. 5B, the spacing between the first and second members 52, 54 is such that the first and second members 52, 54 do not intimately engage or contact the respective inner and outer surfaces 38, 40 of the anulus 34. Instead, the first and second members 52, 54 are “loose” relative to the anulus 34. Where provided, the suture 101 (FIG. 5A) or other component is removed.

The first and second members 52, 54 are then drawn toward one another by forcing the coupler 86 distally along the post 84. In this regard, a clamping tool 102 (shown generally in FIG. 5C) can be provided that mechanically engages the coupler 86 and the post 84, and drives the coupler 86 along the post 84 via movement of handle grips 104. Alternatively, the surgeon can employ other tools and/or manually force the coupler 86 along the post 84. Regardless, the coupler 86 locks (i.e., cannot proximally retract relative to the post 84) at each successively engaged notch 88 (FIG. 4). As the first and second members 52, 54 are forced toward one another, the respective anulus contact faces 60, 74 engage the inner and outer surfaces 38, 40 of the anulus 34. In other words, the anulus 34 is sandwiched between the first and second members 52, 54, and is pinched therebetween. This pinching or compressive force secures the first and second members 52, 54 relative to the anulus 34 in the region of the defect 44. Once desired engagement between the first and second members 52, 54 and the anulus 34 has been achieved, the cap 58 is secured to the second member 54 as shown in FIG. 3.

By preferably configuring the occlusion device 50, and in particular the connector 56, such that a spacing between the first and second members 52, 54 is adjustable, the occlusion device 50 can be used with a variety of different thickness anuli. Further, due to the relatively rigid construction of the first and second members 52, 54, as well as the relatively rigid engagement with the anulus 34, the occlusion device 50 resists undesirable migration or explant of the first member 52 back through the defect 44. In this regard, it should be understood that following the deployment procedure, the disc space 30 will be subjected to normal loads. In response to these loads, the disc space 30 imparts a pushing force on the first member 52 (i.e., transverse force relative to a length of the first member 52, indicated by an arrow in FIG. 3). Construction of the occlusion device 50 in conjunction with pinched engagement of the first and second members 52, 54 to the anulus 34 prevents this pushing force from ejecting the first member 52 back through the defect 44.

When employed as part of a prosthetic spinal disc nucleus implantation procedure, the occlusion device 50 provides the further advantage of resisting not only displacement of the occlusion device 50 itself, but also of the previously-implanted prosthetic spinal disc nucleus 100. In this regard, the normal loads placed on the disc space 30 may cause the prosthetic spinal disc nucleus 100 to migrate from the position shown in FIGS. 5A-5C back toward the defect 44 through which the prosthetic spinal disc nucleus 100 was initially implanted. Alternatively, the prosthetic spinal disc nucleus 100 can have an entirely different construction (e.g., sized to encompass an entirety of the nucleus cavity 42, highly amorphous, etc.), such that a portion of the prosthesis 100 readily contacts the first member 52 following implant and deployment. Regardless, the occlusion device 50 prevents explant or extrusion of the prosthetic spinal disc nucleus 100 or a portion thereof by not only occluding the defect 44, but also by providing strong structural support in the region of the defect 44 such that the occlusion device 50 resists a transverse force otherwise generated by the prosthetic spinal disc nucleus 100 directly on the first member 52. As a point of reference, occlusion devices for occluding cardiac septal defects are known. However, these cardiac septal occluders are configured for the sole purpose of blocking liquid flow, and provide virtually no transverse force resistance. Thus, septal defect occluders have no usefulness for spinal disc anulus repair. The transverse force resistance provided by the occlusion device 50 of the present invention is at least 10 times, more preferably at least 100 times, that provided by known septal defect occluders. Thus, for example, upon final deployment, the occlusion device 50 of the present invention provides transverse force resistance of at least 10 lbs.-force.

As described in greater detail below, the occlusion device in accordance with the present invention can assume configurations varying from the occlusion device 50 associated with the one preferred embodiment. As a general statement, however, components of the occlusion device 50 can incorporate additional features that facilitate repair of the anulus 34. For example, the first and/or second members 52, 54 can be configured to promote tissue regeneration by incorporating a scaffolding construction that can deliver tissue in-growth promoting materials. Alternatively, the first and/or second members 52, 54 can include perforations that promote anulus tissue in-growth. Further, the first and/or second members 52, 54 can provide a roughened/machined surface (e.g., the anulus contact face(s) 60, 74) to assist in maintaining a desired positioning upon final deployment. Also, the occlusion device 50, or components thereof, can be coated, treated, or formed in such a way as to reduce fibrosis or other tissue formation, especially at the outer face 76 of the second member 54 that is otherwise closest to the dura (not shown) of the spinal cord or other nerve tissue. Conversely, one or both of the anulus contact surfaces 60, 74 can be coated or treated in such a way as to induce tissue in-growth for attachment purposes and/or for anulus healing (e.g., scar formation).

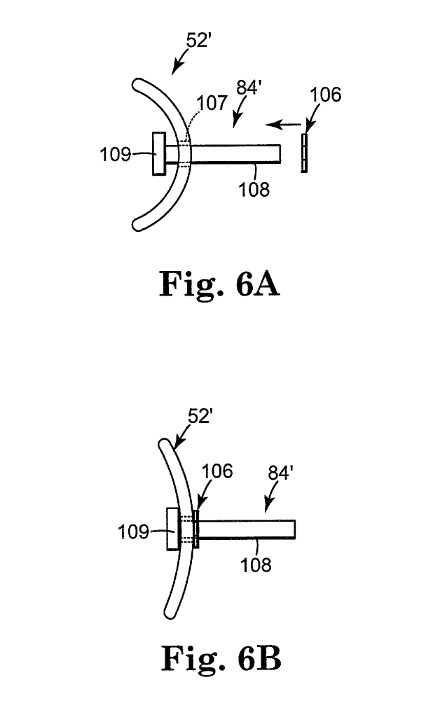

In one alternative embodiment, the first member 52 and the post 84 are provided as separate components to facilitate ease of insertion through the defect or passage 44. For example, FIG. 6A depicts an alternative first member 52′ and an alternative post 84′ in conjunction with a tightening member 106 in a partially assembled state. The first member 52′ is highly similar to that previously described, but is formed of a more flexible material that is biased to a normally folded shape (shown in FIG. 6A) and defines an aperture 107. The post 84′ includes a shaft 108 sized to be received through the aperture 107 and an enlarged base 109. Finally, the tightening member 106 is configured to be received over the shaft 108, and lock thereon. For example, in one preferred embodiment, the tightening member 106 is a speed nut.

Prior to final assembly, the shaft 108 is placed through the aperture 107 and the tightening member 106 is connected thereto as shown in FIG. 6A. However, the base 109 is “loose” relative to the first member 52′ such that the first member 52′ assumes the naturally folded or compressed shape. As such, the first member 52′ is more easily inserted through the annulus defect or passage 44 (FIG. 5A). Once inserted, the tightening member 106 is forced toward the first member 52′, causing the base 109 to press inwardly against the first member 52′. This action, in turn, causes the first member 52′ to unfold or straighten to the deployed position illustrated in FIG. 6B.

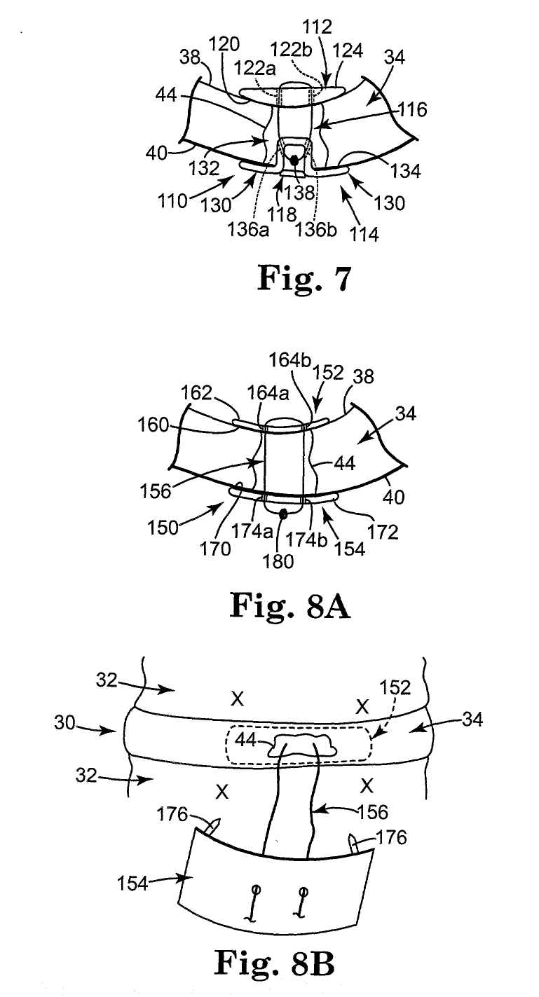

An alternative embodiment occlusion device 110 is provided in FIG. 7, and is illustrated as deployed relative to the anulus 34. The occlusion device 110 is highly similar to the occlusion device 50 previously described, and includes a first member 112, a second member 114, a connector 116, and a cap 118. In general terms, the occlusion device 110 is deployed in a manner similar to that previously described, with the first member 112 engaging the inner surface 38 of the annulus 34, and the second member 114 engaging the outer surface 40 of the anulus 34. The connector 116 connects the first and second members 112, 114, and facilitates forcing the first and second members 112, 114 to the position shown in FIG. 7, as well as securing the components in that position. Finally, the cap 118 is preferably provided to enclose a relevant portion of the connector 116.

The first member 112 is virtually identical to the first member 52 (FIG. 4) previously described, and defines an anulus contact face 120. The first member 112 further forms two passages 122a, 122b that are sized to slidably receive a corresponding portion of the connector 116. In this regard, the passages 122a, 122b preferably extend through an entire thickness of the first member 112 (i.e., the passages 122a, 122b are open at both the anulus contact face 120 and a rear face 124), and are preferably centered relative to a length of the first member 112.

The second member 114 is likewise preferably highly similar to the second member 54 (FIG. 4) previously described, and includes opposing outer regions 130 and a central region 132. The opposing outer regions 130 combine to define an anulus contact face 134. The central region 132 projects distally relative to the opposing outer regions 130, thereby defining an indentation. The second member 114 further forms two passages 136a, 136b in the central region 132. The passages 136a, 136b are positioned to be aligned with the passages 122a, 122b, respectively, of the first member 112 upon final deployment, and are sized to slidably receive a portion of the connector 116.

The connector 116 is a thin, flexible thread (e.g., suture, wire, cable, etc.) that is sized to be slidably received within the passages 122a, 122b, 136a, 136b. Prior to deployment about the anulus 34, the connector thread 116 is threaded through the first passage 136a of the second member 114, through the first passage 122a of the first member 112, around or behind the first member 112, back through the second passage 122b of the first member 112, and finally back through the second passage 136b of the second member 114. To best show this one preferred threading arrangement, the connector thread 116 is illustrated in FIG. 7 as being spaced from an outer face 124 of the first member 112. In practice, however, the connector thread 116 will be tight about the first member 112 upon final deployment.

The above-described arrangement provides for sliding engagement between the connector thread 116 and the first and second members 112, 114. Thus, the connector thread 116 facilitates sliding movement of the second member 114 relative to the first member 112. During use, then, with the first and second members 112, 114 threaded to the connector 116, the second member 114 is retracted relative to the first member 112. The first member 112 is inserted through the defect 44 and into the nucleus cavity 42 (FIG. 5A). The connector thread 116 is available to approximately center the first member 112 relative to the defect 44. The second member 114 is then slid along the connector thread 116 to the final, deployed position illustrated in FIG. 7. In this regard, the surgeon preferably grasps opposing ends of the connector thread 116 proximal the second member 114 to facilitate achieving a tight compression of the first and second members 112, 114 about the anulus 34. Once properly positioned (e.g., the anulus contact face 120 of the first member 112 engaging the inner surface 38 of the anulus 34, and the anulus contact face 134 engaging the outer surface 40 of the anulus 34), a knot 138 is formed in the connector thread 116, thereby securing the occlusion device 110 to the position shown in FIG. 7. Finally, the cap 118 is secured to the second member 114 as previously described. Notably, by preferably extending the connector thread 116 around the first member 112 (i.e., along the rear face 124), the above-described tightening action more easily directs the first member 112 into desired engagement with the inner surface 38 of the anulus 34.

Yet another alternative embodiment occlusion device 150 is shown in FIGS. 8A and 8B in conjunction with the disc space 30 previously described. Once again, the occlusion device 150 includes a first member 152, a second member 154, and a connector 156. As with previous embodiments, the connector 156 rigidly secures the first and second members 152, 154 about the anulus 34 upon final deployment.

The first member 152 is preferably an elongated plate defining an anulus contact face 160, a rear face 162, and passages 164a, 164b. As with previous embodiments, the anulus contact face 160 is preferably generally convex. Further, the first member 152 is sized in accordance with the dimensions previously ascribed for the first member 52 (FIG. 4).

The second member 154 similarly defines an anulus contact face 170, an outer face 172, and passages 174a, 174b. Unlike previous embodiments, the anulus contact face 170 of the second member 154 is substantially continuous (i.e., does not form an indentation), but is generally concave in shape. With additional reference to FIG. 8B, the second member 154 preferably has a height that is greater than an expected height of the anulus 34, and forms distally extending pins 176 at the outer edges thereof. As a point of reference, the view of FIG. 8B illustrates two of the pins 176 disposed adjacent upper comers of the second member 154. It will be understood that in a preferred embodiment, two additional pins disposed adjacent lower comers of the second member 154. With this one preferred embodiment, the pins 176 are configured to anchor the second member 154 into the opposing vertebrae 32. FIG. 8B illustrates the expected contact points between the pins 176 and the opposing vertebrae 32 with an “x”.

Finally, similar to the occlusion device 110 (FIG. 7) previously described, the connector 156 is preferably a thread (e.g., suture) that is slidably received within the various passages 164a, 164b, 174a, 174b. Once again, this preferred configuration allows the first and second members 152, 154 to slide along the connector 156, such that a deployed spacing between the members 152, 154 is adjustable.

During use, following threading of the connector thread 156 to the first and second members 152, 154, the first member 152 is inserted into the nucleus cavity 42 (best shown in FIG. 5A) via the defect 44. The first member 152 is then approximately positioned to the orientation shown in FIG. 8A. The second member 154 is then slid along the connector 156 as shown in FIG. 8B. In particular, the second member 154 is positioned such that the pins 176 contact the adjacent vertebrae 32. The pins 176 are then lodged into the adjacent vertebrae 32, thereby anchoring the second member 154 relative to the disc space 30. Opposing sides 178a, 178b of the connector thread 156 are then simultaneously pulled, drawing the first member 152 toward the second member 154. In particular, the anulus contact face 160 of the first member 152 is directed into engagement with the inner surface 38 of the anulus 34. Once a desired spacing between the first and second members 152, 154 has been achieved (i.e., the anulus 34 being sufficiently pinched between the first and second members 152, 154), a knot 180 is formed in the connector thread 156, thereby securing the occlusion device 150. Notably, while the occlusion device 150 has been described as forming the second member 154 to preferably include the anchor pins 176, these components can be eliminated such that the second member 154 is not mechanically fastened to the adjacent vertebrae 32.

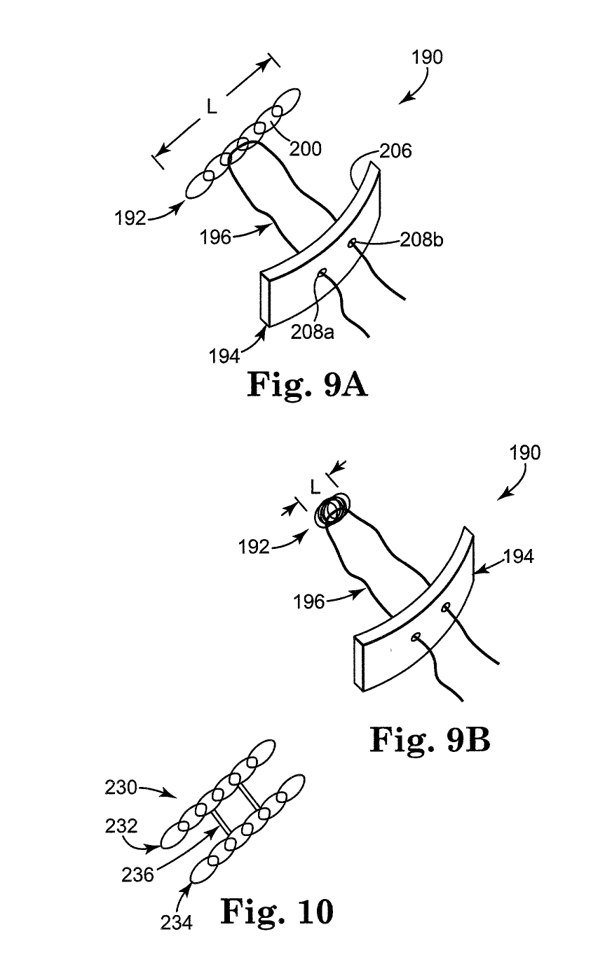

Yet another alternative embodiment occlusion device 190 is provided in FIGS. 9A and 9B. As a point of reference, the occlusion device 190 is shown in FIG. 9A in a final, deployed position, whereas FIG. 9B depicts the occlusion device 190 in a delivery state. With this in mind, the occlusion device 190 includes a first member 192, a second member 194, and a connector 196. In general terms, the first member 192 is configured to be self-expandable from the delivery state (FIG. 9B) to the deployed state (FIG. 9A). The second member 194 is preferably similar to previous embodiments, as is the connector 196. With this general configuration, then, the connector 196 secures the first and second members 192, 194 about the anulus 34 (FIG. 2) upon final deployment.

The first member 192 is preferably a coiled wire (e.g., stainless steel or metal alloy having a shape memory characteristic such as NiTi) that can be longitudinally retracted (i.e., coiled upon itself to provide a reduced overall length “L” in FIGS. 9A and 9B). In the deployed position of FIG. 9A, the first member 192 defines an anulus contact face 200 (referenced generally). In this regard, the first member 192 is preferably configured such that in the deployed state, the first member 192 is relatively transversely rigid. However, the anulus contact face 200 is shaped in accordance with a contour of the individual coils, and is thus not necessarily convex. Alternatively, the first member 192 can assume other forms capable of providing a self-expanding characteristic such that the first member 192 can be contracted to a reduced length prior to deployment. For example, the first member 192 can be a flexible plate that is foldable on to itself (e.g., formed of polyethylene) or as a hydrogel-based component that expands in a predetermined fashion upon imbibing water.

The second member 194 is preferably similar to previous embodiments and defines an anulus contact face 206 and passages 208a, 208b. Thus, the second member 194 is preferably an elongated, relatively rigid plate. Alternatively, the second member 194 can assume the expandable, coil configuration previously described with respect to the first member 192. Finally, similar to previous embodiments, the connector 196 is preferably a flexible thread (e.g., suture) that extends through the passages 208a, 208b, as well as about one or more of the coils provided by the first member 192. Once again, this configuration provides a sliding relationship of the second member 194 relative to the first member 192 along the connector thread 196.

During use, the connector thread 196 is slidably secured to one or more of the coils provided by the first member 192. The first member 192 is then retracted to the delivery state shown in FIG. 9B. With this reduced profile, the first member 192 can be placed within a cannula (not shown) that maintains the first member 192 in the retracted position. The cannula is then inserted through the defect 44 (FIG. 2) such that a distal end thereof is positioned within the nucleus cavity 42. The first member 192 is then released from the cannula and into the nucleus cavity 42. Once released, the first member 192 self-expands from the delivery state of FIG. 9B to the deployed state of FIG. 9A. The connector thread 196 is then slidably connected to the second member 194 via the passages 208a, 208b. Finally, the second member 194 is forced toward the first member 192, and the connector thread 196 secured, as previously described (e.g., a knot is formed). In another alternative embodiment, the preferred configuration of the first and second members (192, 194) are reversed, such that the first member 192 is a relatively rigid plate and the second member 194 has a self-expanding construction.

While previous embodiments have described the occlusion device as including separately formed first member, second member, and connector components, a unitary structure can alternatively be provided. For example, FIG. 10 illustrates an alternative embodiment occlusion device 230 that includes a first member 232, a second member 234, and a connector 236. The first and second members 232, 234 are preferably similar to the first member 192 (FIGS. 9A and 9B) previously described, and are each configured to be self-expanding from a delivery state (not shown) to a deployed state shown in FIG. 10. Thus, in one preferred embodiment, the first and second members 232, 234 are coiled wire that can be longitudinally retracted (i.e., coiled upon itself) to provide a reduced overall length. The connector 236 is rigidly connected at opposite ends thereof to the first and second members 232, 234. In this regard, the connector 236 can be a rigid ring or post that establishes a permanent spacing between the first and second members 232, 234. Alternatively, the connector 236 can include a flexible component (e.g., a thread) that is secured to the first and second members 232, 234 in conjunction with a spacer component (e.g., a ring) that establishes a minimum spacing between the first and second members 232, 234.

Regardless of the exact design, the occlusion device 230 is provided as a unitary structure prior to deployment. That is to say, prior to deployment, the first and second members 232, 234 are permanently attached to at least a portion of the connector 236. The contractible or retractable nature of the first and second members 232, 234 facilitates placement of the entire occlusion device 230 within a cannula (not shown) that otherwise maintains the first and second members 232, 234 in the retracted position prior to deployment. The cannula is then inserted through the anulus defect 44 (FIG. 2) such that a distal end thereof is positioned within the nucleus cavity 42 (FIG. 2). The occlusion device 230 is then directed distally such that the first member 232 is released from the cannula and into the nucleus cavity 42. Once released, the first member 232 self-expands to the deployed state of FIG. 10. The cannula is then removed from the nucleus cavity 42, such that the second member 234 is released from the cannula and self-expands to the deployed state of FIG. 10, at an outside of the annulus 34. The connector 236 establishes a spacing between the first and second members 232, 234, with the occlusion device 230 being secured to opposite sides of the anulus 34 (FIG. 2) in the region of the defect 44.

The spinal disc anulus occlusion device and related method of use of the present invention provides a marked improvement over previous designs. Unlike conventional techniques of suturing an anulus plug or flap to the anulus, the present invention establishes a strong mechanical closure/barrier to the anulus defect. This barrier maintains its position relative to the anulus in response to the normal loads placed upon the disc space, and thus will not unexpectedly dislodge. When provided in conjunction with a prosthetic disc nucleus implant device, the occlusion device of the present invention stabilizes the anulus defect and prevents extrusion or expulsion of the implanted prosthesis.

Although the present invention has been described with reference to preferred embodiments, workers skilled in the art will recognize that changes can be made in form and detail without departing from the spirit and scope of the present invention.

What is claimed is:

1. An occlusion device for occluding a defect in a spinal disc anulus having an interior surface and an exterior surface, and defines an internal cavity, the device comprising:
a first member configured for deployment within the internal cavity and placement against the interior surface of the anulus;
a second member configured for placement against the exterior surface of the anulus; and
a connector for connecting the first and second members, the connector configured to provide an adjustable spacing between the first and second members;
wherein the device is configured such that upon final deployment, the first and second members are secured against the anulus, at opposite surfaces thereof, in a region of the defect via the connector.

2. The device of claim 1, wherein the first member has a height corresponding with a height of the anulus.

3. The device of claim 1, wherein the first member has a height of less than 15 mm.

4. The device of claim 1, wherein the first member is elongated upon final deployment, having a length in the range of 10-30 mm.

5. The device of claim 1, wherein upon final deployment, the first member defines an anulus contact face for contacting the interior surface of the anulus, the anulus contact face being generally convex.

6. The device of claim 5, wherein upon final deployment, the second member defines a second member anulus contact face for contacting the exterior surface of the anulus, the second member anulus contact face being generally concave.

7. The device of claim 1, wherein the second member is slidable relative to the first member along the connector.

8. The device of claim 7, wherein the connector includes a thread slidably receiving the second member.

9. The device of claim 8, wherein the first member defines, upon final deployment, a leading face for contacting the anulus and a trailing face opposite the leading face, and further wherein the thread extends through the leading face to the trailing face.

10. The device of claim 1, wherein the first member, the second member, and the connector are configured as a unitary structure.

11. The device of claim 1, wherein the first member is configured to be self-expanding from a delivery state to a deployed state, a length of the first member being greater in the deployed state than in the delivery state.

12. The device of claim 1, wherein the first and second members are configured to be transversely rigid upon final deployment.

13. The device of claim 1, wherein at least one of the first and second members is a plate.

14. The device of claim 1, wherein the second member defines an inner, anulus contact face and an outer face opposite the inner face, and further wherein a central portion of the outer face defines an indentation.

15. The device of claim 14, wherein the connector includes a locking component for rigidly defining a maximum spacing between the first and second members, and further wherein the indentation is sized to receive the locking component.

16. The device of claim 15, wherein the connector further includes a post extending from an anulus contact face of the first member, the post configured to receive the locking component.

17. The device of claim 15, wherein the connector further includes a thread extending distally from the locking component at the second member to the first member.

18. The device of claim 15, wherein the second member is rigid.

19. A method of occluding a defect in a spinal disc anulus including an inner surface and an outer surface, and defining an internal cavity, the method comprising:
deploying a first member within the internal cavity defined by the spinal disc anulus;
deploying a second member at the outer surface of the anulus in a region of the defect;
extending a connector through the defect, the connector connecting the first and second members; and
securing the first member relative to the second member via the connector such that the first and second member engage the inner and outer surfaces, respectively, of the anulus and rigidly resist transverse expulsion of the first member through the defect.

20. The method of claim 19, wherein securing the first member relative to the second member includes:
guiding the second member toward the first member following deployment of the first member.

21. The method of claim 20, wherein the second member is slidable along a portion of the connector, and further wherein guiding the second member toward the first member includes:
sliding the second member along the connector.

22. The method of claim 21, wherein securing the first member relative to the second member further includes:
locking the connector to establish a permanent maximum spacing between the first and second members.

23. The method of claim 19, wherein securing the first member relative to the second member includes pinching the anulus between the first and second members.

24. The method of claim 19, wherein the first member is a relatively rigid plate, and further wherein deploying the first member includes directing the first member through the defect.

25. The method of claim 19, wherein the first member is configured to be self-expanding from a contracted state to a deployed state, and further wherein deploying the first member includes:
placing the first member in the contracted state;
inserting the first member, in the contracted state, through the defect; and
allowing the first member to expand to the deployed state following insertion.

26. A method of implanting a prosthetic spinal disc nucleus into a nucleus cavity defined by an anulus having an inner and outer surface, the method comprising:
creating an opening through the anulus;
inserting a prosthetic spinal disc nucleus into the nucleus cavity through the opening;
deploying a first occlusion member into the nucleus cavity in a region of the opening;
deploying a second occlusion member at the outer surface of the anulus in the region of the opening;
extending a connector through the opening, the connector connecting the first and second members; and
securing the first member relative to the second member via the connector such that the first and second members engage the inner and outer surfaces, respectively, of the anulus and rigidly resist expulsion of at least a portion of the prosthetic spinal disc nucleus back through the opening.
